(19) United States
(12) Patent Application Publication
(10) Pub. No.: US 2010/0128843 A1
(43) Pub. Date: May 27, 2010

(54) Device for Locating a Female Breast for Diagnostic Imaging and Intervention

(75) Inventor: Ralf Tita, Hirschhorn/Neckar (DE)

Correspondence Address: DAFFER MCDANIEL LLP, P.O. BOX 684908, AUSTIN, TX, 78768, US

(73) Assignee: MIR MEDICAL IMAGING RESEARCH HOLDING GMBH, Moehrendorf (DE)

(21) Appl. No.: 12/401,906

(22) Filed: Mar. 11, 2009

(30) Foreign Application Priority Data
Nov. 22, 2008 (DE) 102008058587.4

(51) Int. Cl. A61B 6/04; H05G 1/00

(52) U.S. Cl. 378/37; 378/208

(57) ABSTRACT

A device for fixing or locating a breast of a female patient in a medical diagnostic examination instrument comprises an elastic annular-shaped fixing means and also a net fastened thereto which tightly encircle a base of a breast close to a chest wall. A force exerted exerted on the elastic annular-shaped fixing means is applied in a direction away from the patient in order to compress the breast and raise the breast from the chest wall. An X-ray machine including such a device is also provided.

### PRIORITY CLAIM

This application claims priority to pending German Application 102008058587.4 filed on Nov. 22, 2008.

### BACKGROUND OF THE INVENTION

1. Field of the Invention

The invention relates to a device for locating, i.e. fixing, a breast of a female patient in a diagnostic instrument, which is for use in diagnostic imaging and interventional therapy. A device of this kind serves for fixing a breast in a position that is advantageous and reproducible in an imaging diagnostic examination. Moreover, this fixing can be used also for taking a tissue sample (biopsy), or for another kind of diagnosis of a tissue under suspicion, or for a therapy.

2. Description of Related Art

Various instruments such as X-ray machines or also CT scanners are known for examining a female breast. A CT scanner of this kind is disclosed for example in U.S. Patent Application Publication No. 2006/0094950. An X-ray facility with a rotating gantry which has an X-ray tube and a detector is located below a patient table on which a patient to be examined rests. A breast of the patient to be examined projects through an opening in the patient table and into a ray path of the X-ray facility. In order to create constant conditions during an examination, the breast to be examined is pushed upwards with a pushing implement and put into a predefined shape. An adaptation to various breast sizes is possible by shifting the implement. However, owing to compression of the breast an examination close to a breast wall is not possible. Another device for stabilizing a breast of a patient is disclosed in U.S. Pat. No. 6,418,188. A cup of rubber-like fabric is positioned to cover a breast, and is then drawn away from the patient by means of a cord. The breast is thereby compressed along a direction of its diameter and elongated. With this device an exactly reproducible position and shape of a breast cannot be achieved. By means of vacuum fixation systems as disclosed in European Publication No. 1864611 a breast is drawn into a bowl-shaped vessel by sub-pressure. This sub-pressure can affect the distribution of liquid within the tissue. Here too, in the case of hard vessels, an invasive diagnostic or therapeutic measure, such as taking a tissue sample, is not possible. The size of the bowl must be adapted to that of a breast, which makes handling difficult.

### BRIEF SUMMARY OF THE INVENTION

The following description of the objective of the disclosure provided herein and the descriptions of embodiments of devices for fixing or locating a breast of a female patient in a reproducible position during a diagnostic examination and/or an invasive therapy and an apparatus comprising such a device is not to be construed in any way as limiting the subject matter of the appended claims.

The objectives of the devices and systems described herein include fixing or locating a breast of a female patient in a reproducible position during a diagnostic examination and/or an invasive therapy, and releasing the breast as far away as possible from a chest without adversely affecting the position and representation of tissue structures.

An embodiment of a device for fixing or locating a breast of a female patient in an instrument for medical examination includes an elastic annular-shaped fixing means which tightly encircles a base of a breast adjacent a chest wall, wherein a force exerted on the elastic annular-shaped fixing means to tightly encircle the base of the breast is along a direction away from the patient.

Another embodiment of device for fixing or locating a breast of a female patient in an instrument for medial examination includes an annular elastic band and a net fastened to a periphery of the annular elastic band, wherein the device is configured constrict the annular elastic band and the net to tightly encircle a breast disposed within the net.

An embodiment of an X-ray machine for imaging a breast of a female patient includes a patient table and an annular elastic band disposed within a cut-out portion of the patient table, wherein the X-ray machine is configured constrict the annular elastic band to tightly encircle a breast disposed within the cut-out potion. The X-ray machine further includes an X-ray tube disposed beneath the patient table and positioned to direct an X-ray beam toward a region directly beneath the cut-out potion of the patient table. Moreover, the X-ray machine includes an X-ray detector disposed beneath the patient table and in a position to receive the X-ray beam generated from the X-ray tube.

### BRIEF DESCRIPTION OF THE DRAWINGS

In the following, the invention is described by way of example, without limitation of the general inventive concept, on examples of embodiments and with reference to the drawings.

FIG. 1 shows an example of a device for fixing or locating a breast in an instrument for medial examination.

FIG. 2 shows an example of a device for fixing or locating a breast of a female patient in an instrument for medial examination, which is particularly flexibly adjustable.

FIG. 3 shows a plan view of an example of a breast fixation or locating device.

FIG. 4 shows a detail of the plan view of the breast fixation or locating device depicted in FIG. 3.

FIG. 5 shows alternative embodiment of a breast fixation or locating device.

While the invention is susceptible to various modifications and alternative forms, specific embodiments thereof are shown by way of example in the drawings and will herein be described in detail. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION OF PREFERRED EMBODIMENTS

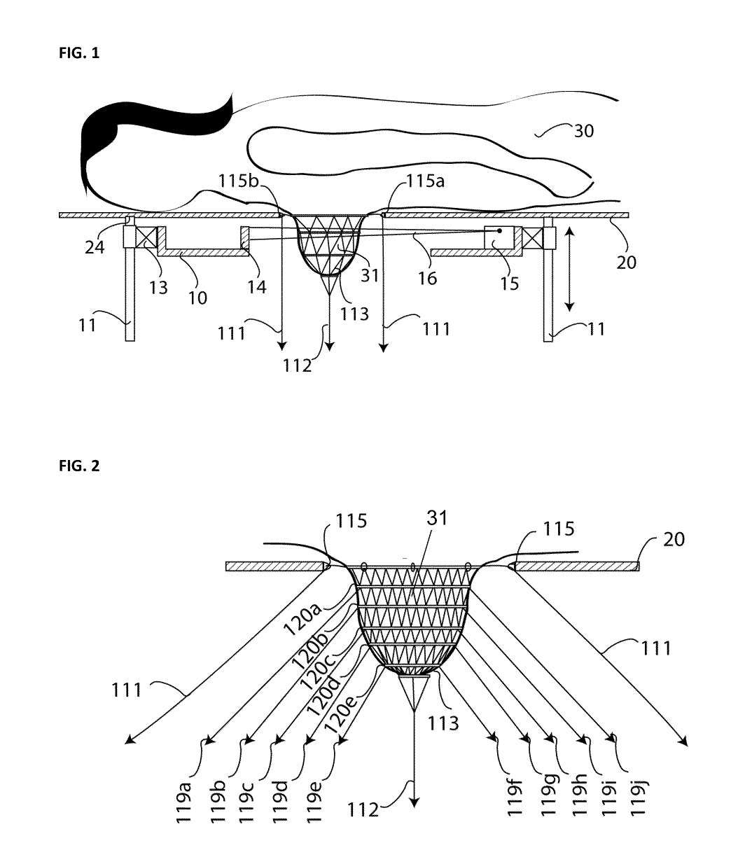

FIG. 1 illustrates a side view of a cross-section through a device for fixing or locating a breast of a female patient in an X-ray machine. A patient 30 is supported on a patient table 20. A spiral computer tomography gantry 10 is located below the patient. A breast 31 of the patient is suspended through a breast cut-out portion of the patient table into an exposure region of the gantry. Within a gantry housing 24, the gantry 10 has an X-ray tube 15 which generates a fan beam 16. Radiation of this fan beam penetrates the breast 31 and is intercepted by a detector 14. For imaging the entire breast, the gantry can be rotated via a gantry pivot bearing 13. Simultaneously with the rotation, a movement of the gantry in direction towards or away from the patient, here in a vertical direction, is effected by a gantry lift drive 11, so that the breast is scanned along a spiral-shaped track. The breast is fixed or located in a fixing or locating device which comprises at least one fixing circle and a net 113. The size of the at least one fixing-circle can be reduced by means of fixing-loop draw-cords 111. Eyelets 115a and 115b are provided within the breast cut-out portion of the patient table for guiding the fixing-loop draw-cords 111. Furthermore, the net 113 can be drawn away from the patient with a net draw-cord 112.

FIG. 2 illustrates a device for fixing or locating a breast of a female patient in an instrument for medial examination, which can be adjusted particularly flexibly. Here, additionally provided tightening cords 119a-j are each fastened to the net and pass around the circumference of the breast. Thereby, draw-rings 120a-e are formed. By pulling a draw-cord along the direction of the arrow, the circumference of the associated draw-ring and, thus, also of the net can be reduced and the pressure on the breast thus increased. Thus, for example, simultaneous pulling of the cords 119a and 119j will reduce the circumference of the net at the draw-ring 120a close to the patient. A reduction of the circumference of the draw-ring 120e close to the nipple is permitted by the draw-cords 119e and 119f. The circumference of the intermediately located draw-rings 120b, 120c, 120d can be changed with the other draw-cords 119b and 119i; 119c and 119h; 119d and 119g illustrated here. In this FIG. 2, pairs of draw-cords are illustrated by way of example. However, basically any other number of draw-cords may be used. Similarly, it is possible to use for one draw-ring, instead of a pair of draw-cords, any other number of draw-cords, for example one draw-cord or also four draw-cords.

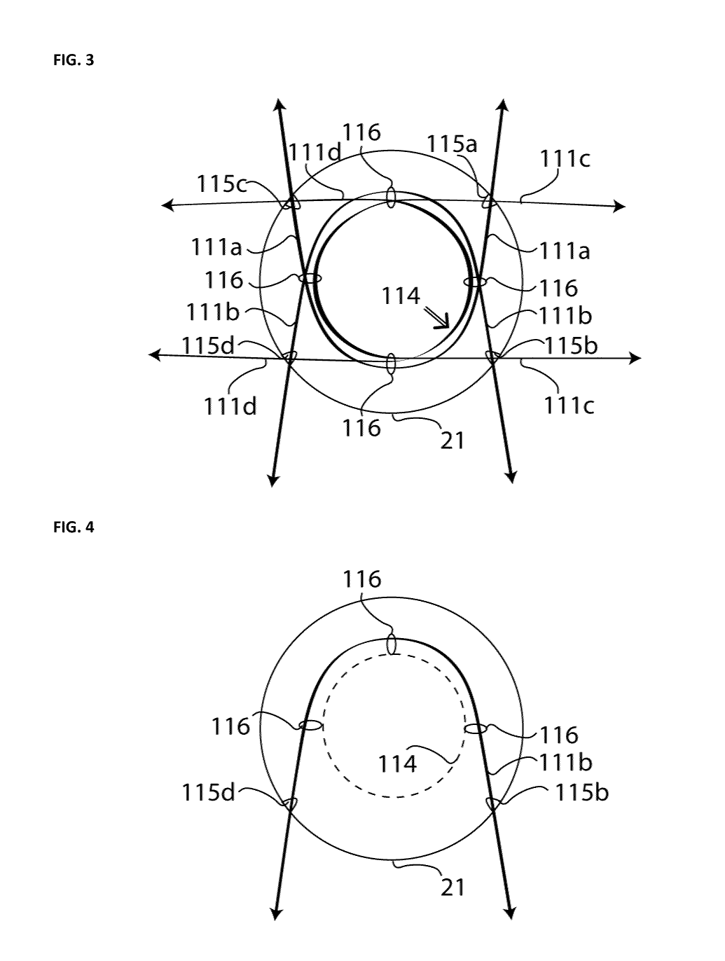

FIG. 3 shows a plan view of a breast fixing or locating device, as shown in FIG. 1 and FIG. 2. Eyelets 115a, 115b, 115c, and 115d for deflecting cords are provided within the breast cut-out portion 21 of the patient table. These guide the fixing-loop draw-cords 111a, 111b, 111c, and 111d. The fixing-loop draw-cords are preferably made of an elastic material which, however, is so rigid that each cord will maintain the approximate shape of the cords as illustrated even under a no-tension condition. Furthermore, rings 116 are provided for holding together the various fixing-loop draw-cords. These fixing-loop draw-cords form an approximate circle 114. Instead of the four fixing-loop draw-cords as illustrated here, any other number of fixing-loop draw-cords is also possible. For example, in some embodiments a single fixing-loop draw-cord may be employed. In such cases, the single fixing-loop draw-cord must enclose the entire breast. In some cases, it may be advantageous to use two fixing-loop draw-cords which are disposed on opposite sides of the breast. The device as illustrated here with four fixing-loop draw-cords makes possible a particularly good fixation and a uniform pressure of the cords against the breast.

For fixing or locating a breast, the fixing-loop draw-cords are drawn in arrow direction as a first step, so that the diameter of the circle 114 is adapted to the size of the breast and closely encloses it. In a second step, the net attached to the fixing-loop draw-cords is drawn away from the patient.

FIG. 4 shows a detail of the plan view of the breast fixing or locating device. Here only one fixing-loop draw-cord 111b is illustrated. The other fixing-loop draw-cords have been omitted for the sake of clarity of representation. The fixing-loop draw-cord 111b is passed around the breast through the rings 116. Other fixing-loop draw-cords are also passed through the rings 116, so that they stabilize each other. Instead of the rings 116, any other desired connecting members may be used which loosely hold together the individual cords. It is also possible to attach further draw cords to these rings for pulling away these rings from a breast. Thereby, preliminary tension can be exerted on the fixing-loop draw-cords. Therewith furthermore, the entire device can be opened in a simple manner as soon as tension from the fixing-loop draw-cords ceases to exist. In order to attain an automatic opening, tension springs could operate on these rings, so that they are drawn outwards away from a breast. In order to achieve defined guidance of the fixing-loop draw-cords, further deflecting eyelets 115b and 115d are provided. These enable, for example, a deflection of the fixing-loop draw-cords away from the patient and out of the operating range of the gantry. A downward deflection of this kind is illustrated in FIG. 1.

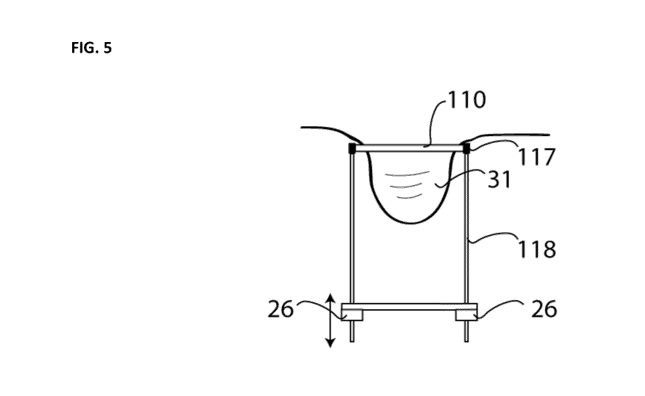

FIG. 5 shows another variant of a breast fixation or locating device. Here, a breast 31 is surrounded by an elastic fixing ring 110. This fixing ring comprises, for example, an elastic synthetic resin material or also a rubber material. The elastic fixing ring 110 is fastened to a positioning unit which here comprises two guide rods 118 and a lift drive 26. Furthermore, at least one force sensor 117 is provided for monitoring a tension of the elastic fixing ring, or a force exerted on the ring, so that no excessive force is exerted on the breast. For fixing the breast, the elastic fixing ring 110 is drawn together, i.e. tightened in a first step, so that it adapts to the diameter of the breast and firmly encloses it. Then, in a second step, this elastic fixing ring 110 is drawn away from the patient by means of the positioning unit. Here too, the force may be monitored. It is possible to perform the drawing-together or the drawing-away from the patient until a maximum force has been attained. Another alternative would be a regulation of constancy of force, in which, for example, the lift drive 26 would exert a constant force on the breast. In any case, the device depicted in FIG. 5 may, in some embodiments, include a net over the breast which may be drawn further enclose the breast, as similarly described for the first embodiment. In this case, the net itself is advantageously drawn in a direction away from the patient.

A device for fixing or locating a breast of a female patient in a medical diagnostic instrument comprises at least one elastic annular-shaped fixing means (e.g., 110 or 114). This encircles a base of a breast adjacent to a chest wall. Furthermore, this fixing involves a force being exerted in a direction away from the patient. Thereby, the breast can be slightly raised from the chest wall. Thus, it becomes more easily possible to cover the entire range of the breast, for example with an X-ray machine.

In some embodiments, a net 113 is provided which is fastened to the elastic annular-shaped fixing means (e.g., 110 or 114). This net also tightly encircles the remainder of the breast. Here, a force is now exerted on the net in a direction away from the patient. Thus, tension results on the net itself and on the elastic annular-shaped fixing means connected therewith.

It is advantageous for the elastic annular-shaped fixing means (e.g., 110 or 114) to comprise a plurality of fixing-loop draw-cords. These may be adjusted individually and in, some cases, are disposed at an angle of 90 degrees to each other. In this manner, fixing-loop draw-cords which are oppositely disposed in pairs can be tightened against each other.

In some embodiments, at least one motor is provided for drawing together and tightening the elastic annular-shaped fixing means (e.g., 110 or 114).

In addition or alternatively, at least one motor may be provided for drawing the elastic annular-shaped fixing means (i.e., 110 or 114) and/or the net 113 away from the patient.

In any case, it may be particularly advantageous for the motor to be connected to a force measuring and control means which switches off the motor when a predetermined force has been attained. As an alternative to this, the motor may be controlled via a regulating loop so that it exerts a constant force.

It is noted that the breast fixation or locating device described herein may be incorporated within an X-ray machine and, and in particular embodiments, to a CT scanner.

It will be appreciated to those skilled in the art having the benefit of this disclosure that this invention is believed to provide devices for fixing or locating a breast of a female patient in an instrument for medial examination and systems including such a device. Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. A device for fixing or locating a breast of a female patient in an instrument for medial examination, comprising an elastic annular-shaped fixing means for tightly encircling a base of the breast adjacent a chest wall, wherein a force exerted on the elastic annular-shaped fixing means to tightly encircle the base of the breast is along a direction away from the patient.

2. The device according to claim 1, further comprising a net fastened to the elastic annular-shaped fixing means, wherein the net is configured to:
tightly encircle a remainder of the breast; and
compress the breast and raise the breast away from the chest wall by a force exerted on the net in a direction away from the patient.

3. The device according to claim 1, wherein the elastic annular-shaped fixing means comprises a plurality of fixing-loop draw-cords.

4. The device according to claim 1, further comprising at least one motor for drawing together and tightening the elastic annular-shaped fixing means.

5. The device according to claim 4, further comprising a force measuring and control means connected to the at least one motor, wherein the force measuring and control means switches off the motor when a predetermined force has been attained.

6. The device according to claim 4, further comprising a force measuring and control means connected to the at least one motor, wherein the force measuring and control means exerts a constant force with the motor via a regulating loop.

7. The device according to claim 1, further comprising at least one motor which draws at least one of the elastic annular-shaped fixing means and the net away from the patient.

8. A device for fixing or locating a breast of a female patient in an instrument for medial examination, comprising:
an annular elastic band; and
a net fastened to a periphery of the annular elastic band, wherein the device is configured constrict the annular elastic band and the net to tightly encircle a breast disposed within the net.

9. The device according to claim 8, wherein the annular elastic band comprises one or more fixing-loop draw-cords each at least partially wrapped around a central axis of the device forming a circle to receive a breast.

10. The device according to claim 9, further comprising one or more rings for holding the one or more fixing-loop draw-cords in the circle, wherein end portions of the one or more fixing-loop draw-cords extend out from the circle.

11. The device according to claim 8, wherein the annular elastic band comprises a ring.

12. The device according to claim 8, further comprising one or more tightening cords each having:
a portion circumferentially arranged and fastened to the net; and
at least one end portion extending from the net.

13. The device according to claim 8, further comprising a net draw cord fastened to an end portion of the net opposing the annular elastic band.

14. An X-ray machine for imaging a breast of a female patient, comprising:
a patient table;
an annular elastic band disposed within a cut-out portion of the patient table, wherein the X-ray machine is configured constrict the annular elastic band to tightly encircle a breast disposed within the cut-out potion;
an X-ray tube disposed beneath the patient table and positioned to direct an X-ray beam toward a region directly beneath the cut-out potion of the patient table; and
an X-ray detector disposed beneath the patient table and in a position to receive the X-ray beam generated from the X-ray tube.

15. The X-ray machine according to claim 14, wherein the X-ray machine is a CT scanner.

16. The X-ray machine according to claim 14, further comprising a net fastened to a periphery of the annular elastic band and extending below the patient table, wherein the X-ray machine is configured constrict the net to tightly encircle a breast disposed within the net.

17. The X-ray machine according to claim 14, wherein the annular elastic band comprises one or more fixing-loop draw-cords each at least partially wrapped around a central axis of the cut-out potion of the patient table forming a circle to receive a breast.

18. The X-ray machine according to claim 17, further comprising one or more eyelets fastened to the cut-portion, wherein the one or more fixing-loop draw-cords pass through the one or more eyelets.

19. The X-ray machine according to claim 14, wherein the annular elastic band comprises a ring.

20. The X-ray machine according to claim 14, further comprising one or more tightening cords each having:
a portion circumferentially arranged and fastened to the net; and
at least one end portion extending from the net.
